(19) United States
(12) Patent Application Publication
(10) Pub. No.: US 2006/0041165 A1
(43) Pub. Date: Feb. 23, 2006

(54) Continuous ethyl lactate preparation method

(75) Inventors: Serge Tretjak, Rouhling (FR); Elie Burtin, Longeville-Les-Saint-Avold (FR); Remy Teissier, Francheville (FR)

Correspondence Address: ARKEMA INC.;PATENT DEPARTMENT - 26TH FLOOR, 2000 MARKET STREET, PHILADELPHIA, PA, 19103-3222, US

(73) Assignee: Arkema,, Puteaux (FR)

(21) Appl. No.: 10/537,422

(22) PCT Filed: Dec. 5, 2003
(86) PCT No.: PCT/FR03/03598

§ 371 Date: Jun. 2, 2005

(30) Foreign Application Priority Data
Dec. 5, 2002 (FR) 02/15348

(51) Int. Cl. C07C 69/68

(52) U.S. Cl. 560/179

(57) ABSTRACT

The invention relates to a continuous method of preparing ethyl lactate by means of lactic acid esterification using ethanol in the presence of a catalyst. The inventive method consists in continuously extracting a mixture comprising ethyl lactate, ethanol, water and different heavy products from an esterification reaction medium at partial lactic acid conversion rate and, subsequently, subjecting said mixture to a reduced-pressure flash separation, thereby producing an overhead stream containing a mixture of ethyl lactate, ethanol and water, the lower part of said mixture supplying a fractional distillation column.

The present invention relates to a process for the preparation of ethyl lactate having a purity of greater than 97% starting from lactic acid or from a lactic acid composition.

Ethyl lactate can be used, alone or in combination with other solvents, as cleaning and degreasing agents, in a washing machine and in a nonaqueous medium, for solid surfaces, such as metal components, ceramics, glass or plastics, which have been contaminated by machining oils or greases and/or for their temporary protection.

It can also be used for the defluxing of printed circuits, which operation consists in removing the soldering flux.

The methods most widely used industrially for producing ethyl lactate consist of an esterification reaction generally catalyzed by acids, according to the reaction:
CH₃CH(OH)CO₂H+EtOH≡CH₃CH(OH)CO₂CH₂CH₃+H₂O (1)

However, the use of this reaction is complicated as a result of the presence of a hydroxyl group on the lactic acid molecule.

Esterification can thus take place between two lactic acid molecules and can then continue to give lactic acid oligomers, according to the following schemes:

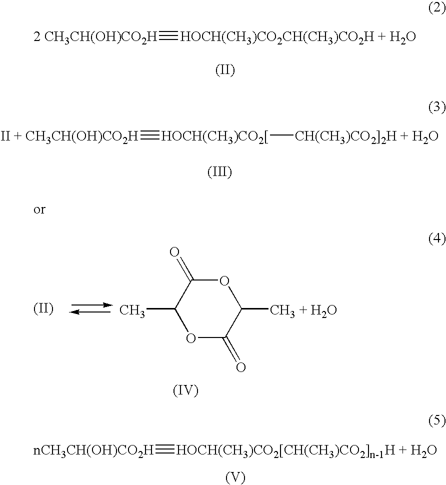

According to the operating conditions generally used, the lactide (IV) is not formed. On the other hand, the oligomers (II), (III) and/or (V) have been detected for the good reason that, industrially, commercial solutions of lactic acid are used.

The term “lactic acid composition” is understood to mean now any aqueous lactic acid solution, whatever its process of preparation and its characteristics, said solution having a highly variable lactic acid purity.

The solutions can in particular be commercially available solutions comprising 50, 80, 87 or 90% of organic compounds, it being understood that such solutions are in fact mixtures of water, of monomers, of dimers and of higher oligomers of lactic acid.

Thus, in order to productively manufacture ethyl lactate (I), it is necessary not only to esterify the lactic acid monomer but also to depolycondense the lactic acid oligomers.

Otherwise, by esterification of the oligomers of lactic acid, oligomers of ethyl lactate are obtained according to the reaction:
CH₃CH(OH)CO₂[CH(CH₃)CO₂]ₙH+C₂H₅OH→CH₃CH(OH)CO₂[CH(CH₃)C(O)]ₙOC₂H₅+H₂O (6)

Consequently, in order to minimize, indeed even eliminate, the formation of the oligomers of ethyl lactate originating from the reaction (6), it is necessary to use a large excess of ethanol and use is generally made of an ethanol/lactic acid molar ratio at least equal to 2.5.

Furthermore, it should be noted that, during the purification of the crude ethyl lactate obtained by esterification of lactic acid with ethanol, a transesterification reaction between two ethyl lactate molecules can occur, according to the reaction:

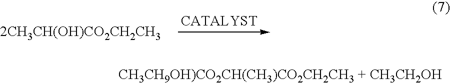

This transesterification reaction (7) is generally carried out in the presence of basic catalysts, of alkyl orthotitanates or of zirconium-based complexes.

Thus, the esterification of lactic acid to give ethyl lactate is rendered more complicated by:
the presence of oligomers of lactic acid in the starting lactic acid compositions, which it is a question of depolycondensing in order to obtain lactic acid,
the competition between the expected esterification (lactic acid, ethanol) and two esterifications which result in the formation of ethyl lactate oligomer (one esterification between lactic acid and ethyl lactate, another between ethanol and an oligomer of lactic acid).

In addition, the Applicant Company has found that it is possible to form a water/ethyl lactate binary azeotrope, thus complicating the removal of the water from the ethyl lactate.

One solution would thus consist in producing, during the esterification of lactic acid by ethanol, an ethyl lactate having a water content as low as possible, in order to subject it to a purification consisting of a distillation under reduced pressure.

Thus it is that the Applicant Company has found that it is possible to obtain an ethyl lactate comprising virtually no more water by continuously extracting from the esterification reaction medium, at a partial degree of conversion of the lactic acid, a mixture comprising ethyl lactate, ethanol, water and heavy products composed of unconverted lactic acid and oligomers of ethyl lactate, by subjecting this mixture to a flash separation under reduced pressure, from which the Applicant Company obtained two streams:
as bottom product, from the flash separation, a stream comprising lactic acid and oligomers (which can advantageously be recycled in the reaction medium);
as top product, from the flash separation, a stream comprising a mixture of ethyl lactate, of ethanol and of water;
   and by then subjecting this top stream to a fractional distillation under certain conditions, from which the Applicant Company obtained an ethyl lactate comprising virtually no more water.

A subject matter of the present invention is thus a continuous process for the preparation of ethyl lactate (I) by esterification of lactic acid [or of a lactic acid composition] using ethanol according to the reaction (1):
CH₃CH(OH)CO₂H+CH₃CH₂OH≡CH₃CH(OH)CO₂CH₂CH₃+H₂O (1)
(I)
which consists in reacting said lactic acid with ethanol according to an ethanol/lactic acid molar ratio at least equal to 2.5 and preferably ranging from 2.5 to 4.5, in the presence of a catalyst, at a temperature ranging from 50° C. to 90° C. and preferably ranging from 80° C. to 90° C., at atmospheric pressure; said process being characterized in that:
a mixture comprising ethyl lactate, unconverted lactic acid, ethanol, water and small amounts of heavy products is continuously extracted, at atmospheric pressure, from the reaction medium at a degree of conversion of the lactic acid at most equal to 80%; then in that
this mixture is subjected to a flash separation at a temperature of between 80° C. and 90° C. and under a pressure of less than or equal to 65 mbar, and in that,
on the one hand, the top stream, comprising ethyl lactate, ethanol and water, is subjected to a continuous fractional distillation, at atmospheric pressure, said stream being introduced onto a specific plate of a distillation column;
on the other hand, the bottom stream, composed essentially of unconverted lactic acid and of heavy products, is continuously recycled to the esterification reaction medium;
   and in that a mixture of ethanol and of water is recovered as top product from the fractional distillation and an ethyl lactate having a water content at most equal to 0.3%, an ethanol content of less than 0.5% and a purity of greater than 94% is recovered as bottom product from the fractional distillation.

According to the present invention, the mixture is extracted from the reaction medium when a degree of conversion of the lactic acid at most equal to 80% has been reached and preferably when this degree of conversion is between 65% and 75%.

This mixture can be extracted from the stirred reaction medium by simple overflowing and then conveyed to a flash separation device.

The reaction is carried out in the presence of a catalyst which is soluble or insoluble in the esterification reaction medium.

Mention will be made, as examples of soluble catalysts which can be used according to the present invention, of 98% H₂SO₄, H₃PO₄ or methanesulfonic acid.

Preferably, 98% H₂SO₄ will be used.

The catalyst according to the invention is used at molar contents ranging from 0.1% to 4% and preferably at contents ranging from 0.2% to 3%, with respect to the 100% lactic acid employed.

According to the present invention, it is possible to operate in a stirred reactor or using a fixed bed technology. In the latter case, solid catalysts, such as ion-exchange resins of the Amberlyst 15 type, will be used.

According to the present invention, the top stream exiting from the flash separation feeds a fractional distillation column at an appropriate point in said column preferably situated in the bottom part of said column. This point will be determined by a person skilled in the art by the calculation, taking account in particular of the number of theoretical plates of the column, of the reflux ratio, of the desired fractionation. Distillation is carried out at atmospheric pressure at a column bottom temperature ranging from 152° C. to 165° C.

The top products from said distillation comprise ethanol in amounts at most equal to 85% (by weight), water and traces of ethyl lactate. This mixture can be dehydrated and the alcohol, in the azeotropic form, can be recycled in the esterification reaction medium. The ethyl lactate obtained as bottom product from the fractional distillation has a water content at most equal to 0.3% and can be subjected to purification by distillation under reduced pressure (removal of heavy compounds, such as the dimer of ethyl lactate, and of traces of lactic acid).

The process according to the present invention applies very particularly to the esterification by ethanol of the lactic acid present in commercial lactic acid compositions as defined above.

Preferably, lactic acid compositions comprising 87% by weight of lactic acid will be used.

The ethyl lactate originating from the bottom product from the fractional distillation comprises virtually no water or alcohol, which makes it possible to obtain, after easy purification, a pure ethyl lactate.

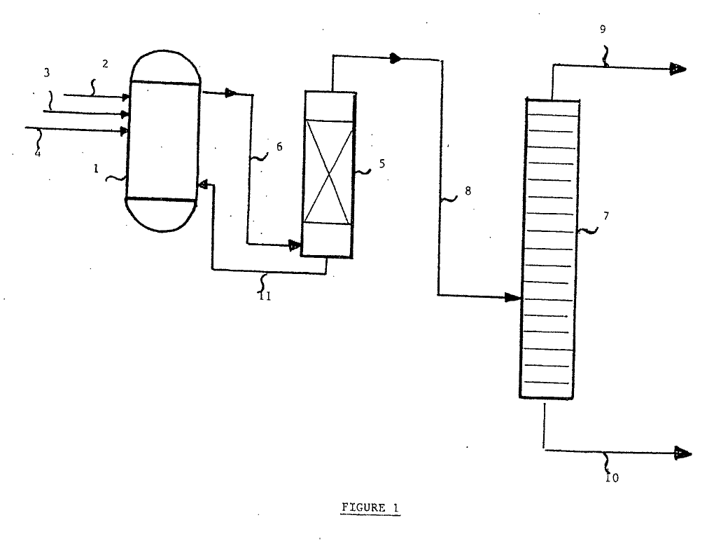

The process according to the present invention can be carried out in a device as represented in FIG. 1.

This device comprises:
a reactor (1), optionally equipped with a stirrer, a temperature probe, a lactic acid feed (2), an ethanol feed (3) and a catalyst feed (4);
a flash separation column (5) fed with phase extracted from the reactor (1) via the feed line (6);
a fractional distillation column (7) fed with top stream from the column (5) via the feed line (8) and equipped with a top outlet (9) for the ethanol-water mixture and with a bottom outlet (10) for the ethyl lactate;
a feed to the reactor of heavy products (11) originating from the bottom of the flash separation column (5).

The example which follows illustrates the invention.

### EXAMPLE

A lactic acid composition comprising 87% by weight of lactic acid is esterified with the device as represented diagrammatically in FIG. 1.

The distillation column (7) has a diameter of 70 cm and is filled with a Sulzer B×70 packing. It has 35 theoretical plates.

Carrying Out the Test

The following are introduced into the reactor (1):
an 87% lactic acid composition,
absolute ethanol,
98% sulfuric acid.

The ethanol/lactic acid molar ratio is equal to 2.5. Esterification is carried out at 80° C. at atmospheric pressure. The progress of the reaction is monitored by quantitative determination of the lactic acid by GC. When the conversion of said lactic acid has reached 70%, a mixture comprising:
ethyl lactate, ethanol, lactic acid and water, is continuously extracted from the reactor (1).

This mixture is subjected to flash separation in the column (5) at 85° C. under a pressure of 50 mbar. The top stream comprising:
ethyl lactate, ethanol, lactic acid and water.

This mixture is subjected to flash separation in the column (5). The top stream, comprising 44% of ethanol, 42% of ethyl lactate and 14% of water, is subjected to fractional distillation in the column (7), which is fed with said top stream at the 13^{th} theoretical plate.

The fractional distillation is carried out at a column bottom temperature of 155° C. The top temperature is 77.2° C. The reflux ratio is set at 1.3. A mixture comprising (by weight) 76% of ethanol, 24% of water and traces of ethyl lactate (<0.3%) is obtained as top product. Ethyl lactate exits as bottom product with a purity of greater than 94.6% and comprising less than 1% of water and less than 1% of ethanol.

This crude ethyl lactate is subjected to purification by fractional distillation under reduced pressure.

1. A continuous process for the preparation of ethyl lactate (I) by esterification of lactic acid using ethanol according to the reaction (1):
CH₃CH(OH)CO₂H+CH₃CH₂OH≡CH₃CH(OH)CO₂CH₂CH₃+H₂O (1)
(I)
comprising reacting said lactic acid with ethanol according to an ethanol/lactic acid molar ratio at least equal to 2.5, in the presence of a catalyst, at a temperature ranging from 50° C. to 90° C., at atmospheric pressure; said process comprising:
continuously extracting a mixture comprising ethyl lactate, unconverted lactic acid, ethanol, water and small amounts of heavy products, at atmospheric pressure, from the reaction medium at a degree of conversion of the lactic acid at most equal to 80%; then
subjecting this mixture to a flash separation at a temperature of between 80° C. and 90° C. and under a pressure of less than or equal to 65 mbar, and
the subjecting a top stream from said flash separation, comprising ethyl lactate, ethanol and water, to a continuous fractional distillation, at atmospheric pressure, said top stream from said flash separation being introduced onto a specific plate of a distillation column; and
continuously recycling a bottom stream, composed essentially of unconverted lactic acid and of heavy products, to the esterification reaction medium; and,
recovering a mixture of ethanol and of water as a top product from the fractional distillation and
recovering an ethyl lactate having a water content which makes possible its subsequent purification as a bottom product from the fractional distillation.

2. The process as claimed in claim 1, characterized in that use is made of an ethanol/lactic acid molar ratio ranging from 2.5 to 4.5.

3. The process as claimed in claim 1, characterized in that the mixture is extracted continuously from the reaction medium when the degree of conversion of the lactic acid is between 65% and 75%.

4. The process as claimed in claim 1, characterized in that the top stream exiting from the flash separation feeds a fractional distillation column at a point situated in the bottom part of said column.

5. The process as claimed in any one of claim 1, characterized in that the fractional distillation of the top stream resulting from the flash separation is carried out at a column bottom temperature ranging from 152° C. to 165° C.

6. The ethyl lactate obtained as claimed in claim 1, characterized in that it has a water content at most equal to 0.3%.

7. The process as claimed in claim 1 characterized in that the temperature of said reaction ranges from 80° C. and 90° C.
